(19) United States
(12) Patent Application Publication
(10) Pub. No.: US 2017/0056485 A1
(43) Pub. Date: Mar. 2, 2017

(54) CRYSTALLIZED XYLOSE ISOMERASE IN PREVENTION OF THE DEVELOPMENT OF NON-ALCOHOLIC FATTY LIVER DISEASE

(71) Applicant: SCIOTEC DIAGNOSTIC TECHNOLOGIES GMBH, Tulln (AT)

(72) Inventor: Albert MISSBICHLER, Vienna (AT)

(21) Appl. No.: 14/841,019

(22) Filed: Aug. 31, 2015

(51) Int. Cl. A61K 38/52

(52) U.S. Cl. CPC A61K 38/52; C12Y 503/01005

(57) ABSTRACT

The present invention relates to the composition comprising crystalline xylose-isomerase and at least one salt of a metal and/or alkaline earth metal for the treatment and prevention of non-alcoholic fatty liver disease and other fructose-related disorders.

### FIELD OF THE INVENTION

The present invention relates to a composition for the treatment of Non-Alcoholic Fatty Liver Disease and the treatment or prevention of fructose-related disorders.

### BACKGROUND

Non-alcoholic fatty liver disease (NAFLD) is a rapidly-growing and mostly silent chronic liver disease characterised by the accumulation of triglycerides in hepatocytes occurring in people who consume little or no alcohol. This condition comprises a wide spectrum of histological lesions ranging from simple steatosis to non-alcoholic steatohepatitis (NASH), a parenchymal liver inflammation which can develop further to fibrosis, cirrhosis and hepatocellular carcinoma. NAFLD is strongly associated with obesity and insulin resistance and is currently conceptualised as the hepatic manifestation of metabolic syndrome (MS). Growing evidence suggests that the fast-growing and alarming epidemic of NAFLD is closely intertwined with the Westernization of dietary patterns with an increasing intake of simple sugars, especially fructose.

Fructose (formerly also called laevulose) is a 6-carbon ketone monosaccharide (simple sugar). Fructose is commonly found in table sugar (sucrose), where it is chemically bound to the 6-carbon ketone monosaccharide glucose. Fructose is the sweetest of all naturally occurring carbohydrates, and is the sugar that imparts sweetness in table sugar. It is a primary component of high fructose corn syrup, which is a reduced glycemic index alternative to table sugar. Free fructose is increasingly used in the food sector because of its sweetness intensity, which is approximately 1.7 times higher than that of table sugar, and its improved transportability.

In contrast to glucose, fructose is not actively absorbed in the intestine, but is passively resorbed by special proteins at a substantially slower rate. Nearly half of the population is not able to resorb more than 25 g of fructose per day. The average daily consumption, however, is between 11 g and 54 g per day. A major part of fructose is consumed with soft drinks, which have an increasing importance in the average food intake. In addition, the increasing use of high fructose corn syrup sweeteners aggravates the problem.

Xylose-isomerase is used on a large industrial scale in the food industries for the preparation of fructose from glucose, in order to enhance the degree of sweetness. Under the physiological conditions in the small intestine (i.e., absorption/retraction of glucose from the equilibrium reaction), xylose-isomerase isomerizes fructose into glucose. Orally-administered xylose-isomerase is able to catalyze the conversion of poorly absorbable fructose into well-absorbable glucose in the human intestine in vivo. The ability of xylose-isomerase to effectively reduce overall fructose intake provides an avenue for the treatment and/or prevention of NAFLD, and other fructose-related disorders, which are correlated with high dietary fructose intake. Fructose not only is obviously present in aforementioned nutrients but also makes 50% of the disaccharide sucrose, which is enzymatically cleaved into the monosaccharides glucose and fructose. Of an ingested sucrose load, only 20% of the glucose but 100% of the fructose is metabolized by the liver. Fructose induces many biophysiological processes such as: 1) substrate-dependent phosphate depletion, which increases uric acid (a major trigger for the development of gout, Pillinger and Abeles 2010) and contributes to hypertension 2) de novo lipogenesis and dyslipidemia; 3) hepatic lipid droplet formation and steatosis; 4) muscle insulin resistance; 5) hepatic insulin resistance, which promotes hyperinsulinemia and influences substrate deposition into fat; 6) central nervous system hyperinsulinemia, which antagonizes central leptin signaling and promotes continued energy intake. Simply put, fructose has a negative impact on human metabolism and lacks the capability of signaling this fact. Fructose must therefore be considered a major health threat in the western community

In DE 102006013624 xylose-isomerase is generally described as a means for the conversion of fructose into glucose. In Bhosale (1996), xylose-isomerase is generally described as a means for the isomerization of fructose into glucose and vice versa. Furthermore, the importance of alkaline earth metal salts for the activity of xylose-isomerase is discussed among others. In WO 03/051391 A1, glucose isomerase and a separate magnesium salt are employed for the treatment of obesity and diabetes. In WO 91/05857A, a method for the crystallization of enzymes, such as glucose-isomerase, is described. Furthermore, this document shows that during the crystallization of enzymes, bivalent salts such as magnesium sulfate may be used. WO 01/12834A relates to a composition which comprises cross-linked crystalline xylose-isomerase and is contacted with magnesium sulfate in the course of the substrate conversion. In U.S. Pat. No. 3,847,740, a composition is disclosed which comprises xylose-isomerase and magnesium carbonate. In WO 03/099410A, a method for the separation and purification of nucleosides is described in which cross-linked crystalline xylose-isomerase is used. In Carrell, the x-ray structure of xylose-isomerase is disclosed. The authors state that bivalent metal ions such as magnesium are required for the catalytic activity of xylose-isomerase. The crystallization of xylose-isomerase is performed according to methods known in the art, e.g., Suzuki et al.; Dunlop and Hazes; Vilonen et al.; Ramagopal et al.

However, none of these references disclose a highly active xylose-isomerase composition. Therefore, there is a need in the industry for a highly active xylose-isomerase composition for the treatment of nonalcoholic fatty liver disease.

### SUMMARY

It has been determined that xylose-isomerase, crystallized in the presence of salts of metal and/or alkaline earth metals, has high activity relative to traditionally-produced xylose-isomerase and therefore is effective in treating non-alcoholic fatty liver disease. Co-crystallization of xylose-isomerase in the presence of specific concentration ranges of metal salts, such as magnesium salt and other bivalent metal salts, delivers a highly active crystalline xylose-isomerase composition. The acidic environment in the stomach, as well as endogenous proteases, may have a detrimental effect on the activity of crystalline xylose-isomerase. Crystalline xylose-isomerase may be formulated in such a way as to help protect the enzyme and its activity. For example, xylose-isomerase may be enterically coated in order to shield the enzyme from degradatory environments. Crystalline xylose-isomerase may also be cross-linked in order to provide linked enzymes with increased stability. Cross-linkage of xylose-isomerase may be accomplished by established methods, e.g. Vallejo-Becerra et al., and Wenzel et al. The stability of cross-linked xylose-isomerase allows it to be administered orally with or without enteric coating.

The xylose-isomerase composition may be in the form of a dried, fine granular powder, which may be crystallized in the presence of metal ions as co-factors in order to ensure rapid bioavailability and high specific activity. The crystals of xylose-isomerase may be finely ground in a mill. This kind of preparation leads to maximum activity in the physiological environment of the intestine and quick release, based in part on high instestinal lumen solubility.

Xylose-isomerase exhibits increased enzymatic activity in presence of metal salts. Bivalent metal salts offer particular utility. A composition comprising xylose-isomerase co-crystallized with at least one metal salt provides a highly active xylose-isomerase composition for administration to a subject. The composition may comprise at least one and up to 15 different metal salts.

Some embodiments of the invention are directed towards a method of treating or preventing non-alcoholic fatty liver disease in a subject. In some embodiments, a method of treating or preventing non-alcoholic fatty liver disease decreases circulating lipid concentrations. In some aspects, the method comprises administering to the subject a composition comprising xylose-isomerase in the presence of at least one metal salt. In particular embodiments, the composition comprises xylose-isomerase that is co-crystallized with at least one metal salt. In some embodiments, the at least one metal salt is a bivalent metal salt. In particular embodiments, the composition comprises xylose-isomerase co-crystallized with a magnesium salt and at least one bivalent metal salt selected from the group consisting of cobalt salt, zinc salt, iron salt, and copper salt. A metal salt co-crystallized xylose-isomerase composition enables high enzymatic activity in the intestinal region, and in particular in the small intestine. The aforementioned metal salts may comprise a metal cation and any counter-ion known to those of skill in the art, including but not limited to a halide, sulfate, carbonate, bicarbonate, fumarate, nitrate, nitrite, sulfite, phosphate, phosphite, phosphonate, tartarate, oxalate, acetate, hexafluorophosphate, benzoate, and benzenesulfonate. In some embodiments, the xylose-isomerase originates from a microorganism of the family Streptomycetaceae. In a particular embodiment, the xylose-isomerase originates from Streptomyces rubiginosus.

In some embodiments, administration of a composition comprising xylose-isomerase co-crystallized with a magnesium salt and at least one bivalent metal salt selected from the group consisting of cobalt salt, zinc salt, iron salt, and copper salt decreases circulating lipid concentrations. In some embodiments, decreasing circulating lipid concentrations comprises decreasing fatty acid, fatty ester, cholesterol, and/or cholesterol esters levels in the blood.

In some embodiments, the composition comprises a molar ratio of the bivalent metal salt selected from the group consisting of cobalt salt, zinc salt, iron salt, and copper salt to xylose-isomerase ranging from 0.1:1 to 100:1, preferably from 0.5:1 to 20:1, more preferably from 3:1 to 7:1. In further embodiments, the composition comprises a molar ratio of the magnesium salt to xylose-isomerase ranging from 0.5:1 to 200:1, preferably from 5:1 to 25:1, more preferably from 12:1 to 18:1. The aforementioned metal salts may comprise a metal cation and any counter-ion known to those of skill in the art, including but not limited to a halide, sulfate, carbonate, bicarbonate, fumarate, nitrate, nitrite, sulfite, phosphate, phosphite, phosphonate, tartarate, oxalate, acetate, hexafluorophosphate, benzoate, and benzenesulfonate. In particular embodiments, the magnesium salt is selected from the group consisting of MgCl₂, MgSO₄, MgCO₃, Mg(HCO₃)₂, or Mg(C₄H₂O₄). In a particular embodiment, the composition comprises magnesium and/or cobalt salts. Most particular are those compositions of the invention which include magnesium as well as cobalt salts.

Some embodiments are directed towards a method of treating hepatic steatosis in a subject comprising administering to the subject a composition comprising crystalline xylose-isomerase. Some aspects of the invention are directed towards a method of reducing liver weight in a subject suffering from hepatic lipid accumulation. Some aspects of the invention are directed towards a method of preventing or reducing obesity of a subject comprising administering to the subject a composition comprising crystalline xylose-isomerase. In some embodiments, the crystalline xylose-isomerase originates from a microorganism of the family of Streptomycetaceae. The crystalline xylose-isomerase may be co-crystallized with a magnesium salt and at least one bivalent metal salt selected from the group consisting of cobalt salt, zinc salt, iron salt, and copper salt. The composition may comprise a molar ratio of the bivalent metal salt selected from the group consisting of cobalt salt, zinc salt, iron salt, and copper salt to xylose-isomerase ranging from 0.1:1 to 100:1, preferably from 0.5:1 to 20:1, more preferably from 3:1 to 7:1. In further embodiments, the composition comprises a molar ratio of the magnesium salt to xylose-isomerase ranging from 0.5:1 to 200:1, preferably from 5:1 to 25:1, more preferably from 12:1 to 18:1. The aforementioned metal salts may comprise a metal cation and any counter-ion known to those of skill in the art, including but not limited to a halide, sulfate, carbonate, bicarbonate, fumarate, nitrate, nitrite, sulfite, phosphate, phosphite, phosphonate, tartarate, oxalate, acetate, hexafluorophosphate, benzoate, and benzenesulfonate. In particular embodiments, the magnesium salt is selected from the group consisting of MgCl₂, MgSO₄, MgCO₃, Mg(HCO₃)₂, or Mg(C₄H₂O₄).

### BRIEF DESCRIPTION OF THE FIGURES

The present invention will be described in more detail with reference to the accompanying drawings and examples, which, however, should not be considered as limitation.

FIG. 1 shows the influence of the pH on the activity of xylose-isomerase.

FIG. 2 shows the acid-stability of xylose-isomerase.

FIG. 3 shows the temperature stability of enteric coated pellets of xylose-isomerase.

FIG. 4 is a graph comparing the activity of crystallized xylose-isomerase to a solution of xylose-isomerase.

FIG. 5 shows the influence of ions of bivalent metals on the activity of xylose-isomerase.

FIG. 6 shows the influence of co-crystallization of xylose-isomerase with bivalent metal ions on the activity of xylose-isomerase.

FIG. 7 shows the in vivo effect of xylose-isomerase in the course of time as compared with a placebo and no administration of a substance.

### DETAILED DESCRIPTION

The co-crystallization of xylose-isomerase in the presence of specific concentration ranges of a magnesium salt and other bivalent metal salts affords a highly active crystalline xylose-isomerase composition, which may be used for the treatment of nonalcoholic fatty liver disease. The acidic environment in the stomach, as well as endogenous proteases, may have a detrimental effect on the activity of crystalline xylose-isomerase. In order to reduce or prevent its degradation, xylose-isomerase may be formulated in such a way as to help protect the enzyme and its activity.

According to a particular embodiment, the crystals of xylose-isomerase are used as fine, dried powder. The powdered form is more stable against bacterial degradation. The powdered form advantages during manufacturing; when pelleting on a large technical scale, improved dosing ability and mixability are observed. The powder form of xylose-isomerase preferably has a residual water content of 0.1% to 30%, more particular of 0.5% to 10% and most particular from 1% to 3%. The protein content of the powder is preferably 50% to 99.9%, more particular 75% to 99.9%, and most particular 95% to 99.9%. The particle size of the powder ranges from 0.01 μm to 1000 μm, preferably from 0.1 μm to 100 μm and most particular from 1 μm to 30 μm.

The xylose-isomerase composition prepared and made available according to the invention is preferably “highly active”. This “highly active” xylose-isomerase composition exhibits an enzyme activity from 35,000 to 45,000 units per gram (total preparation). Here one unit (U) is defined as μmol per gram per hour at 37° C. (35,000 and 45,000 U correspond to 9.72 and 12.5 μ-Katal, respectively (μkat; kat=mol/s)). By contrast, xylose-isomerase from a lyophilisate or directly purified from a column, has an activity from about 4,000 to 6,000 U/g (determined according to Dische et al.), that is, 1.1 to 1.7 μkat.

To ensure sustainable protection of fructose overload in the liver, the xylose-isomerase composition may be administered orally before a meal or drink. In some embodiments, a xylose-isomerase composition is administered before, during, or after a meal or drink. In some embodiments, a xylose-isomerase composition is administered before, during, or after a meal or drink containing at least 10 g of fructose. In other embodiments, a xylose-isomerase composition is administered before, during, or after a meal or drink containing at least 25 g of fructose. The permanent conversion of even small amounts of fructose into glucose triggers the release of leptin and reduces the circulating amounts of ghrelin. This results in a feeling of satiation, which aids in preventing and reducing obesity, a major problem associated with NAFLD.

In certain embodiments, the xylose-isomerase composition may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier.

In some aspects, the composition is administered in an enteric form. The enteric form may be an enteric coated pellet, enteric coated tablet, enteric coated capsule, enteric coated granule, or enteric coated powder. The coating may be applied in an amount ranging from 1% to 50% by weight, based on the total weight of the dosage forms. Methacrylic acid/alkyl(meth)acrylate-copolymers are particular, copolymers of methacrylic acid/methyl-methacrylate having a ratio of 1:1 to 1:2, such as Eudragit L® or Eudragit S®, are more particular and copolymers of methacrylic acid/ethylacrylate 1:1, such as Eudra-git L55®, Eudragit L30D-55®, which quickly dissolve at a pH value of >5.5, are most particular. Enteric coatings based on celluloses or shellac, which are known to the persons skilled in the art, may be applied. Furthermore, materials available as EudraGuard® may be used as enteric coatings. The coatings may be applied with suitable solutions or dispersions, in organic or aqueous medium, with an aqueous medium being particular. The enteric coated dosage forms are preferably also resistant to saliva, with coatings on the basis of Eudragit E or Eudragit EPO being suitable.

The phrase “enteric coated” means a characteristic of a dosage form to protect an active ingredient contained therein (e.g., xylose-isomerase) from the action of gastric juice or a solution having properties comparable to gastric juice (e.g., acid) for a determined time period of at least 10, preferably at least 20, more particular at least 30, most particular at least 60 minutes, in such a way that the active ingredient is subject to a loss of activity of a maximum of 50%, preferably a maximum of 40%, more particular a maximum of 30%, most particular a maximum of 20%, and in particular a maximum of 10%. The particular dosage forms are prepared by mixing the starting materials with the enzyme preparation, granulating, extruding, dividing and optionally shaping, preferably spheronizing, optionally classifying, and providing them with an enteric coating.

Enteric coated pellets are pellets enveloped with an enteric coating, which dissolve at a pH value as present in the intestinal tract, i.e., such coatings preferably dissolve at a pH value of at least 4 and maximum 10. Eudragit, for example, is an enteric coating based on anionic polymers of methacrylic acid and methacrylates, contains the —COOH functional group, and dissolves in the range of pH 5.5 to pH 7. As an alternative to Eudragit, shellac or acetylated starch, e.g., Amprac 01 may be used. As the enteric coatings known in the state of the art have different properties, e.g., dissolution rate and pH at which the coating dissolves, the materials of the coatings may be combined as well. Shellac, for example, exhibits good acid resistance but dissolves very slowly in the intestinal tract. Amprac 01, on the contrary, dissolves quickly in the intestinal environment, but does not have a sufficient acid resistance. In order to compensate for the drawbacks of a material, both of the above mentioned materials may be mixed, for example, in a weight ratio of 60-95/40-5, preferably of 70-90/30-10 of shellac/Amprac 01. Another parameter influencing the release rate of the active in gredient is the layer thickness of the enteric coated pellet. The layer thickness, expressed as a mass ratio, is preferably 5 to 30%, more preferably 10 to 20% of the total mass of the final product. The pellets preferably have an average diameter of 0.5 to 5 mm, in particular of 0.7 to 2 mm. Such a pellet size allows the pellet to pass through the stomach quickly.

The preparation of the pellets, which allows for the use of the crystalline xylose-isomerase composition as a drug, food supplement, dietetic food, medicinal product, feeding stuff, supplementary feeding stuff or dietetic feeding stuff, is made preferably using an extruder. The extrusion process calls for thermal stability of the ingredients of the composition, in particular of the enzymes, up to 60° C. (Stricker). The pellets may include further pharmaceutical ingredients, in addition to the enzymes and enteric coating. For example, microcrystalline cellulose, e.g., Avicel, serves as filler and swelling agent. Cellulose is insoluble in water and comprises crystalline as well as amorphous fractions. This combination causes a plastic deformability, i.e., an irreversible change in shape occurs if sufficient force is applied. This is important for pelleting in the extruder and spheronizer. During wet granulation, microcrystalline cellulose absorbs large quantities of water and becomes an easily compressible, cohesive mass, even in the absence of binding agents. The amount of microcrystalline cellulose in a pellet may be added in an amount ranging from 5 to 70%, preferably between 10 and 60%, and more preferably between 15 and 50%.

Maltose may be used as binding agent and filler. Maltose enhances the solubility of the matrix and therefore assists in the rapid release of the enzyme. Maltose may be added in an amount ranging from 1 to 40%, preferably 5 to 35%, and more preferably 10 to 30%. As compared to saccharose, maltose has the advantage that it does not include any fructose, and therefore does not introduce any unnecessary and therefore harmful fructose into the body. Hydroxypropyl cellulose may also be added as binding agent, and aids in reducing fine dust. In addition, hydroxypropyl cellulose increases the strength of the pellets and therefore helps to improve yield. Hydroxypropyl cellulose may be added in a particular amount ranging from 0.5 to 10%.

Starch may be added as filler and disintegrant in a particular amount ranging from 1% to 30%. As a water-insoluble substance, starch may absorb large amounts of water and is therefore an ideal disintegrant. Cross-carmellose (Na-CMC; Acdisol) is a pure disintegrant which preferably may be added in an amount ranging from 1% to 5%. High Acdisol content may causes early disintegration of the pellets during spheronizing. Crosspovidon, a cross-linked polyvinylpolypyrrolidone (PVP), is also insoluble in water and also serves as disintegrant. As a result of its polymeric characteristics, it promotes improved spheronizing during the preparation of pellets. Crosspovidon may be added preferably in an amount ranging from 0.5 to 10%. Povidon is a water-soluble additive and serves as binding agent. Combinations of these different fillers, disintegrants and binding agents results in a molecular disperse distribution of xylose-isomerase in the pellet and promotes advantageous bioavailability.

An isolating layer consisting of glycerine and/or talc may be provided subjacent to the enteric coating. Glycerine acts as an humectant that prevents loss of moisture and subsequent inactivation of the enzyme.

As particles having a diameter of more than 3 mm may trigger an occlusion reflex at the pylorus, it is particular that the enteric coated dosage forms, including pellets, leave the stomach with a size of less than 3 mm. Particles less than 3 mm in diameter may pass the pylorus in the closed state and may be transported like liquid from the stomach into the small intestine. The neutral pH value prevailing there promotes pellet disintegration within about 5 to 30 min, preferably 15 min, and thus releases the active substances. Therefore, it is particular to provide pellets with a diameter of less than 5 mm, preferably of less than 3 mm, as enteric coated dosage forms.

As an alternative to pellets, xylose-isomerase may also be transported in capsules or other dosage form through the stomach into the intestinal tract. Suitable capsule formulations include, but are not limited to, gelatine capsules and starch capsules. The capsules may also contain pellets comprising a xylose-isomerase composition.

In some embodiments, the composition is present in microcapsules, nanoparticles, or liposomes. The composition may be provided as a pharmaceutical composition, a food supplement, a dietetic food, a medicinal product, a feeding stuff, a supplementary feeding stuff or a dietetic feeding stuff. Any dosage form which is known to those of skill in the art may be employed.

“Gastric juice” means the natural composition of the gastric juice as well as artificial gastric juice preparations (pH 1-2), well known to the persons skilled in the art. The term “release in the small intestine” is meant to comprise the release in the natural juice of the small intestine, as well as the release in preparations similar to the juice of the small intestine at pH values of 6-7.5, preferably pH 6.4-6.8.

In some embodiments, the enzyme or enzymes may be of different origins. According to particular embodiments, xylose-isomerase is of microbial, animal, vegetable, or recombinant origin. Methods for the isolation and/or preparation of the enzymes are well known to those skilled in the art. In particular embodiments, xylose-isomerase of microbial origin is employed. Xylose-isomerase from microbial origin may originate from a microorganism of the family of Streptomycetaceae, in particular Streptomyces rubiginosus. Xylose-isomerase from sources of this kind have higher specific activity on glucose/fructose and smaller Km than isomerases of other sources. For example, xylose-isomerase derived from Lactobacillus brevis has a Km of 920 nM, whereas xylose-isomerase derived from Streptomyces rubiginosus has a Km of 160 nM.

The composition of the invention may be used in the form of different products. Preferably the composition is a pharmaceutical composition, a food supplement, a dietetic food, a medicinal product, a feeding stuff, a supplementary feeding stuff or a dietetic feeding stuff.

In some aspects, a composition comprising crystalline xylose-isomerase (EC 5.3.1.5) and at least one salt of a metal and/or alkaline earth metal is provided. The composition may be provided in an enteric coated dosage form, which is in particular selected from the group consisting of enteric coated pellet, enteric coated tablet, enteric coated capsule, enteric coated granules and enteric coated powder. In some aspects, there is a method for the preparation of a drug comprising xylose-isomerse for the treatment of non-alcoholic fatty liver disease. In some embodiments, the drug is provided in enteric coated dosage form.

In some embodiments, a composition comprising xylose-isomerase is taken immediately prior to, or with each meal containing fructose in order to aid fructose isomerization. As particles having a diameter of more than 3 mm trigger an occlusion reflex at the pylorus, it is particular that the enteric coated dosage forms leave the stomach with a size of less than 3 mm. Particles with a size of less than 3 mm may pass through the pylorus in the closed state and may be transported like liquid from the stomach into the small intestine. The neutral pH value prevailing in the small intestine facilitates pellet disintegration within about 5 to 30 min, preferably 15 min, and releases the active components.

In one aspect, there is a method for the preparation of a composition comprising the step of crystallizing xylose-isomerase in presence of at least one salt of an alkaline earth metal and/or metal, where the crystalline xylose-isomerase is dried and optionally powdered, and formulated as pharmaceutical dosage form, in particular selected from the group consisting of enteric coated pellet, enteric coated tablet, enteric coated capsule, enteric coated granules and enteric coated powder. Drying is preferably performed in vacuum and is subdivided into a) filtration of the crystals from the solution and subsequent b) freeze-drying of the filter cake. In some embodiments, the residual moisture of the filter cake is less than 5%. In some aspects, the dry filter cake is ground, for example using a corn mill.

The industrially-used enzyme contains large amounts of interfering substances, which inhibit the activity of the enzyme in a decisive way. Sorbitol is one of the major interfering substances found in industrial xylose-isomerase. The sorbitol content of the crystalline xylose-isomerase of the invention is <1%. Due to its low sorbitol content, the composition displays notably increased activity over its industrial counterpart. Mannose-isomerase may alternatively be used for the conversion of fructose into mannose. Mannose is also absorbed in the small intestine, as explained above, and therefore also retracted from the equilibrium reaction.

### Example 1

The effects of oral administration of enteric coated pellets containing crystallized xylose-isomerase on the histopathological and pathogenic features of non-alcoholic fatty diver disease (NAFLD) induced by high fructose diet were examined. Xylose-isomerase pellets were prepared according to the following procedures.

### Xylose-Isomerase pH Dependency

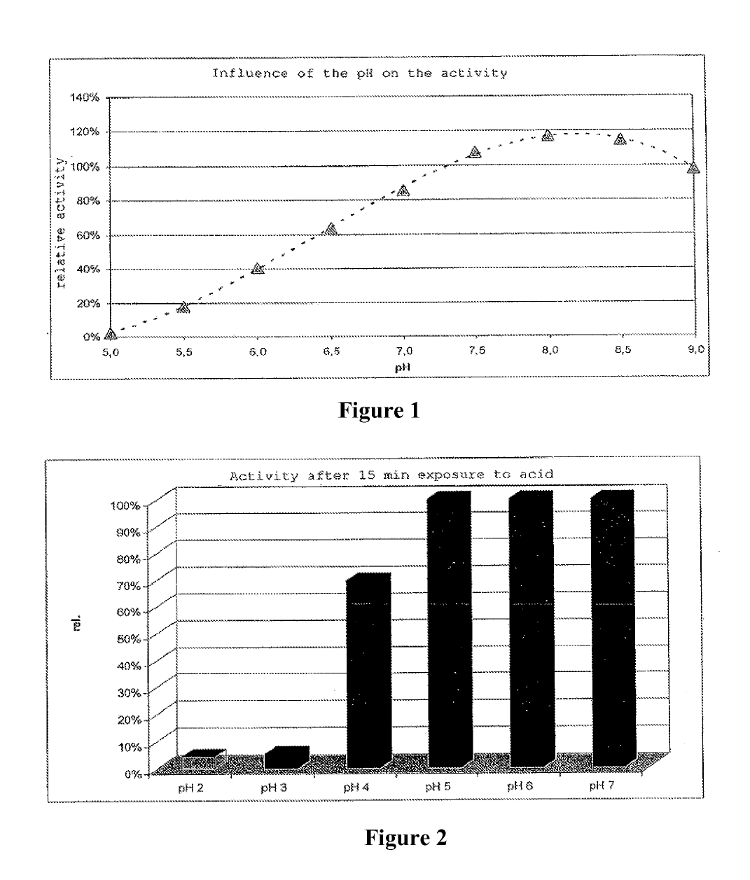

In order to ensure bioavailability in the intestinal region of an individual or animal, it is advantageous to transport xylose-isomerase from gastric juice into the small intestine in protected form. The determination of the pH-dependency of the activity of the enzyme (FIG. 1) and the stability of the enzyme at certain pH values (FIG. 2) serve as the basis for the development of a suitable formulation. The pH-dependency of the activity of the glucose isomerase was measured in 50 mM maleate, hepes or Tris-buffer/pH 5 to pH 9, with 5 mM MgSO₄, 1 mM CoCl₂ and 100 mM glucose at 37° C. The fructose obtained was measured with a modified sulfuric acid/carbazole-test according to Dische and Bohrenfreud. In order to examine stability, a defined amount of enzyme was incubated in 50 mM glycine, maleate or hepes-buffer at a pH of 2 to 7 at 37° C. for 30 min. The buffer was neutralized with an excess of 100 mM hepes buffer, pH 7.4, and the activity was measured in presence of 5 mM MgSO₄, 1 mM CoCl₂ and 100 mM glucose at 37° C. The fructose obtained was measured with a modified sulfuric acid/carbazole-test according to Dische and Bohrenfreud.

### Xylose-Isomerase Temperature Stability

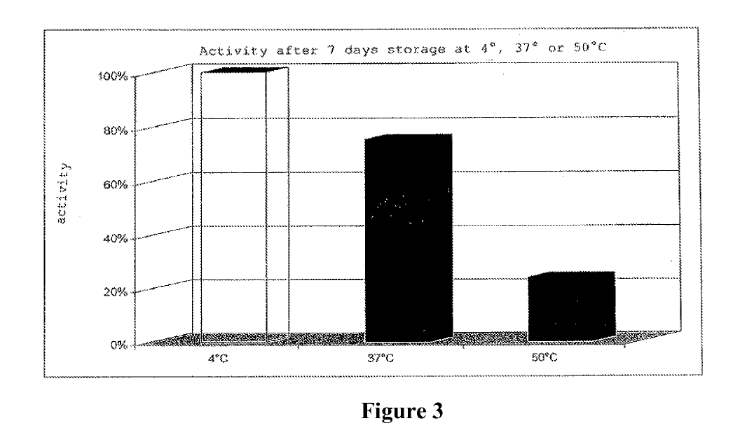

The stability of xylose-isomerase pellets is of great importance for storage and sale. Sufficient stability is valuable for providing a market-acceptable product. Coated pellets were stored at 4° C., 37° C. and 50° C. for 7 days and the activity was measured under standard conditions. After 7 days at 37° C., the activity decreased by 25% and after 7 days at 50° C. by 77% (FIG. 3).

### Comparison of Crystalline and In-Solution Xylose-Isomerase

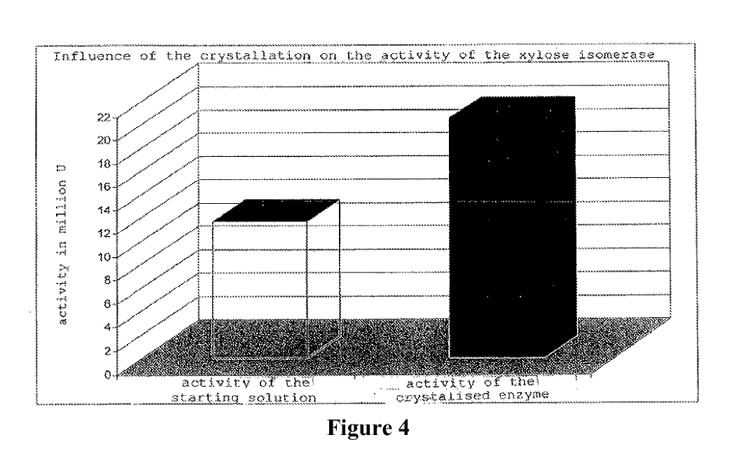

Xylose-isomerase was obtained from Streptomyces rubiginosus. This xylose-isomerase was purified by a crystallization process employing sorbitol and other additives. Crystalline xylose-isomerase displays an increase in activity of 70% over a solution-based xylose-isomerase composition (FIG. 4).

### Influence of Bivalent Metal Ions Added Post-Crystallization

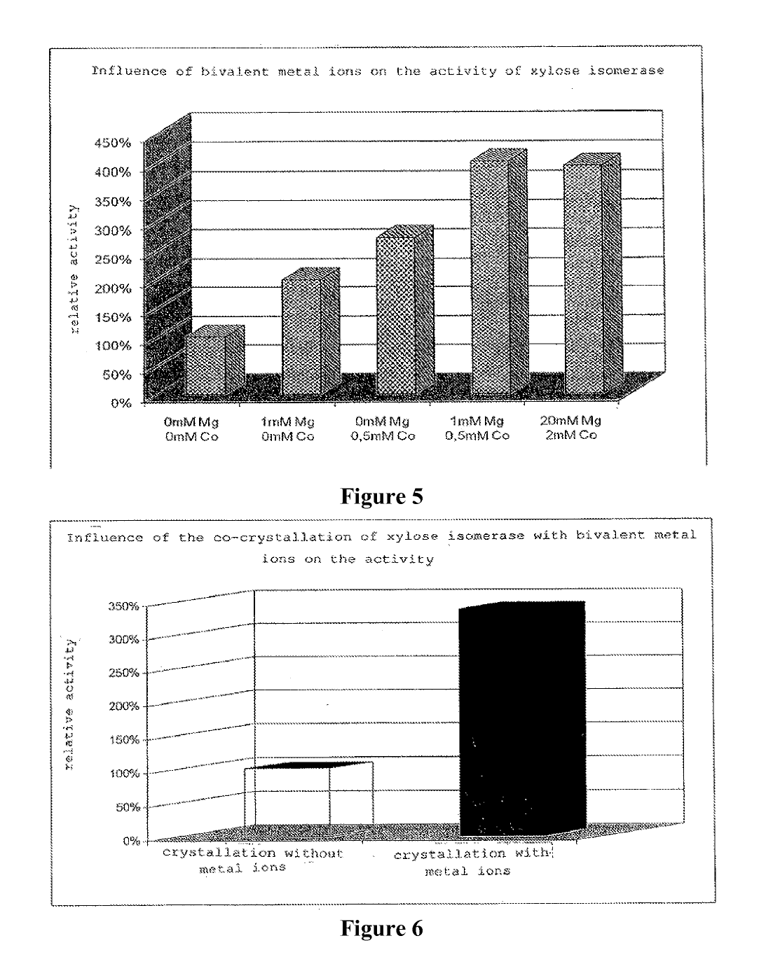

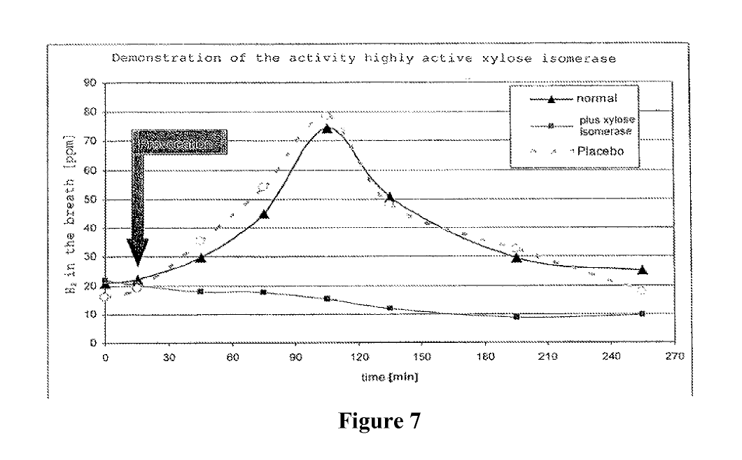

Different concentrations of Mg²⁺ and Co²⁺ were added to xylose-isomerase purified without addition of bivalent metal ions. Xylose-isomerase activity was measured in 50 mM phosphate buffer, pH 7.4, and 100 mM glucose at 37° C. An increase in enzyme activity of up to 300% was observed for the two compositions comprising both Mg²⁺ and Co²⁺ cations (FIG. 5).

### Influence of Co-Crystallization of Xylose-Isomerase With Bivalent Metal Ions

Xylose-isomerase was crystallized in the presence and absence of Mg²⁺ and Co²⁺. The activities of the compositions were measured in 50 mM phosphate buffer, pH 7.4, with 100 mM glucose at 37° C. Compared to the enzyme crystallized in the absence of bivalent metal ions, the relative activity of the crystals co-crystallized in the presence of bivalent metal ions was 300% (FIG. 6).

### Crystallization of Xylose-Isomerase in the Presence of Co-Factors

To 5 liters of a 4% w/v xylose-isomerase solution was added 735 g ammonium sulfate, 72 g magnesium sulfate-hexahydrate and 19.4 g cobalt(II)chloride-hexaydrate. The solution was slowly cooled down to 2° C. and stirred for 20 hours. In order to accelerate the crystallization process, 50 ml of a 4% xylose-isomerase crystal suspension may be added. The resulting crystals should have an optimum size between 50 and 100 μm. This procedure nearly corresponds to the crystallization in U.S. Pat. No. 4,699,822, however, CoCl₂ and starter crystals are added.

### Drying of Xylose-Isomerase Crystals

The xylose-isomerase crystal suspension obtained from the Xylose-Isomerase pH Dependency experiment, depicted above, is filtered through a pleated filter of class 3 hw (Sartorius, Germany). The crystals obtained are frozen and lyophilized. For optimum processability the solid enzyme cake may be finely ground. The xylose-isomerase powder obtained has an average activity of 45,000 units per gram. One unit of xylose-isomerase is defined as the enzymatic activity, which converts 1 μmol (180 μg) of glucose into fructose in 50 mM phosphate buffer, pH 7.4, with 5 mM MgS04, 1 mM CoCl2 and 100 mM glucose at 37° C. per hour (standard conditions).

### Preparation of Xylose-Isomerase Pellets

a) Granulation: 31.4 g hydroxypropyl cellulose, 408.7 g microcrystalline cellulose, 169.5 g rice starch, 15.6 g croscarmellose, 62.2 g crospovidon, and 145.3 g maltose were mixed with 167.1 g xylose-isomerase powder (7,500,000 units). The mixture of solids was processed with 377 g of bidistilled water into a wet, crumbly mass. This mass was extruded into strands through a sieve having a pore size of 1 mm (Caleva Extruder 10/25, Caleva Process Solutions Ltd.)

b) Spheronization: The wet strands were rolled into pellets in a spheronizer (Spheronizer 250, Caleva Process Solutions Ltd.) at 400 rpm for 5 minutes. The pellets were then dried on racks at 35° C. until the weight was constant.

c) Classification: The dried pellets were classified in a sieve tube, and the fraction having a size in the range from 0.4 to 0.8 was selected for further processing.

d) Characterization of the pellets: The total pellet yield was 70%. The release test in the dissolution tester resulted in a release of 85.8% of the activity within 5 minutes, and 97.5% of the input activity within 15 minutes into the surrounding environment. Rapid release of the enzyme enables precipitous kinetics in the gastrointestinal tract after peroral administration.

e) Pellet Coating: Based on its protein structure, xylose-isomerase is inactivated in the stomach primarily by pepsin and the acidic pH. Therefore, protection of the enzyme by an enteric coating or an enteric coated anionic matrix is desired for the preservation of the enzymatic activity.

Coating solution: 1.9 kg acetylated starch was dissolved in 152 kg purified water while stirring (suspension 1). Using an Ultraturrax, 10.9 kg shellac SSB 63 Hydram was stirred in additional 100 kg purified water to obtain a solution (suspension 2). The suspensions 1 and 2 were mixed together and 0.6 kg glycerol 85% and 2.6 kg micronized talc were added. During the spraying procedure the coating solution was stirred with an Ultraturrax.

f) Coating: 1 kg of pellets was coated with 1.5 kg coating solution in a fluidized bed. The device parameters were selected as follows: spray pressure 1.6 bar, spray rate 180 g/minute, inlet air temperature 55° C., product temperature: 35° C.; inlet air quantity 1400 m³/h. After application of the complete spraying solution, the pellets were re-dried at an inlet air temperature of 40° C. for 60 minutes.

g) Product Characterization: The coated pellets met the test for gastric resistance according to the Pharmacopoea Europaea. After two hours of incubation in the disintegration tester at 37° C. in 0.1 N hydrochloric acid, the pellets were unchanged. An exchange of the medium to phosphate buffer pH 6.8 caused the disintegration of the enteric coated pellets within one hour. The coating amounted to approximately 16% of the original weight. The pellets may be used as preliminary product for tablets and capsules.

### Effects of Crystallized Xylose-Isomerase Pellets on Non-Alcoholic Fatty Liver Disease

The effects of oral administration of enteric coated pellets containing crystallized xylose-isomerase on the histopathological and pathogenic features of non-alcoholic fatty liver disease (NAFLD) induced by high fructose diet were examined. The objective was to explore whether orally-administered xylose-isomerase pellets in Sprague Dawley rats fed ad libitum a high fructose diet affect serum activity of liver enzymes, liver histology and liver levels of pro-inflammatory cytokines. The study was performed with use of 6 high fructose diet fed (4 males per group) and 6 conventional diet fed (2 males per group) groups of Sprague-Dawley (SD) rats.

### Rat Diets

I. High Fructose Rat Diet plus 48 mg/g XI-Pellets (HF-RAT DIET+XI-Pellets)

II. Standard Rat Diet plus 48 mg/g XI-Pellets (STANDARD RAT DIET+XI-Pellets)

III. High Fructose Rat Diet (HF-RAT DIET)

IV. Standard Rat Diet (SD)

TABLE 1
Administration Route, Frequency, Dose Volume
Route of Administration, Frequency and Dose Volume

| | |
|---|---|
| Route | Via the diet; administration the test item the same route as that |
| | [symbol] |
| Volume | Ad libitum |
| Duration | 5 weeks + 5 weeks; very highfructose diet induces significant |
| | [symbol] |

[symbol]

indicates data missing or illegible when filed

Six groups of SD rats were treated with enteric coated pellets containing crystallized xylose-isomerase pellets and 6 groups with placebo mixed in the food for 5 weeks. After 5 weeks, 2 groups of xylose-isomerase pellet fed (one high fructose diet fed and one conventional diet fed) and 2 of placebo (one high fructose diet fed and one conventional diet fed) were sacrificed and livers were harvested. For the next 5 weeks, 2 groups of xylose-isomerase pellet fed (one high fructose diet fed and one conventional diet fed) and 2 of placebo (one high fructose diet fed and one conventional diet fed) were fed the same diet, and 2 groups of xylose-isomerase pellet fed (one high fructose diet and one conventional diet) and 2 of placebo (one high fructose diet and one conventional diet) were switched.

TABLE 2
Subject Groups
Groups

| | Total | ID | | Total | ID |
|---|---|---|---|---|---|
| Group | [symbol] | [symbol] | Group | [symbol] | [symbol] |
| HF-Rat Diet/Weeks 1-5 | | | Standard Rat Diet/Weeks 1-5 | | |
| XI-pellets 1 | 4 | 1-4 | XI-pellets 4 | 2 | 25-26 |
| XI-pellets 2 | 4 | 5-8 | XI-pellets 5 | 2 | 27-28 |
| XI-pellets 3 | 4 | 9-12 | XI-pellets 6 | 2 | 29-30 |
| Placebo 1 | 4 | 13-16 | Placebo 4 | 2 | 31-32 |
| Placebo 2 | 4 | 17-20 | Placebo 5 | 2 | 33-34 |
| Placebo 3 | 4 | 21-24 | Placebo 6 | 2 | 35-36 |
| HF-Rat Diet/Week 6-10 | | | Standard Rat Diet/Week 6-10 | | |
| XI-pellets 1 | 4 | 1-4 | XI-pellets 4 | 2 | 25-26 |
| XI-pellets P2 | 4 | 17-20 | XI-pellets P5 | 2 | 33-34 |
| Placebo 1 | 4 | 13-16 | Placebo 4 | 2 | 31-32 |
| Placebo XI2 | 4 | 5-8 | Placebo XI5 | 2 | 27-28 |

[symbol]

indicates data missing or illegible when filed

Body weight and food consumption were recorded daily. Clinical observations were recorded daily, and detailed observations were recorded weekly. Blood samples for clinical chemistry examinations were collected before treatment, after week 5, and thereafter at the end of treatment. Liver samples were collected after week 5 in selected animals, and week 10 in the remaining animals. Liver samples were divided into three parts, for different analyses.

TABLE 3
Animal Observations

| Procedure | Frequency of Testing |
|---|---|
| Cage side Observation (mortality, moribund) | Daily |
| Clinical Observation | Weekly |
| Body Weight | Daily |
| Food Consumption | Daily |

### Blood Sampling

Blood samples were collected from fasted animals; clinical chemistry parameters were analysed at the same day, as blood was collected. The serum for insulin evaluation was frozen at −20° C. until the analysis. Processing of blood and determination of clinical chemistry parameters were performed according to standard operating procedures.

TABLE 4
Blood Sampling

| | | Animal | Total | | | Sampling | |
|---|---|---|---|---|---|---|---|
| Time | Groups | ID | No | Volume | Tubes Used | Site | Anaesthesia |
| Begin | XI-pellets 1-6 | 1-36 | 36 | ≈1 mL | Serum | Retro-bulbar | yes |
| | | | | | Separator | Venoplex | |
| Week 5 | XI-pellets 1-6 | 1-36 | 36 | ≈1 mL | Serum | Retro-bulbar | yes |
| | | | | | Separator | Venoplex | |
| Week 10 | XI-pellets1 | 1-4 | 24 | ≈1 mL | Serum | Retro-bulbar | yes |
| | XI-pellets P2 | 17-20 | | | Separator | Venoplex | |
| | Placebo 1 | 13-16 | | | | | |
| | Placebo XI2 | 5-8 | | | | | |

Liver Tissue Homogenization Procedure: Liver specimens were taken immediately after the rats were sacrificed and frozen at −70° C. until analyses.

Determination of UAC and Fe: Liver specimens (0.5 g) were homogenized with a homogenizer in a 10-fold volume of saline (5 mL). After homogenization, tissue homogenates were centrifuged at 10,000×g for 5 min at 4° C. The supernatants were frozen at −20° C. until analyses.

Determination of cytokines and leptin: Liver specimens (0.5 g) were homogenized with homogenizer (low speed for ˜20 seconds) in 5 mL PBS with 25 μL of protease inhibitors (solution chilled on wet ice). Homogenate samples were centrifuged at 10,000×g for 5 minutes. The supernatant was used for measurement of cytokines and leptin. Total proteins in supernatant were determined according to standard operating procedures.

Quality Control: The quality control measurement of clinical chemistry parameters were assured by the control serum Lyonorm Human N. External control measurement of clinical chemistry parameters were assured by external quality control (SEKK Ltd., Bartolomejska 90, 530 02 Pardubice, Czech Republic, provider of proficiency testing schemes no. 7004 accredited by CAI).

Pathology: At the end of week 5, designated animals were sacrificed, and at the end of week 10, the remaining animals were sacrificed by application of an overdose of anaesthetic (diethyl ether), and livers were harvested.

During necropsy, the liver of each rat was weighed. Livers were subsequently divided into three parts, for different processing methods. The Lobus caudatus (pars retroventricularis) underwent histopathological examinations, lobus sinister lateralis sections were employed for UAC and Fe evaluations, and remnant liver lobes were used to determine cytokines and leptin. Each part of the liver was weighed separately.

A sample of liver tissue intended for histopathological processing was divided into two parts. The first part was fixed in formalin, the second in bouin fixative. The liver slices were stained by hematoxylin and eosin (HE) method and Azan by Heidenhain for the proof of fibrosis. Steatosis, inflammatory lesions, necrosis, hyperaemia and fibrosis in the liver slices were evaluated by light microscope. Histopathological lesions were quantified on the basis of four characteristics: no lesion, slight, small, and medium. The autopsy and histological examinations were performed according to standard operating procedures.

TABLE 5
Autopsy
Autopsy

| | | | Total | Speci- | |
|---|---|---|---|---|---|
| Time | Groups | Animal ID | No | men | Histology |
| Week 5 | XI-pellets 3 | 9-12 | 12 | Liver | Azan & |
| | Placebo 3 | 21-24 | | | Haematoxylin/ |
| | XI-pellets 6 | 29-30 | | | Eosin Stains |
| Week 10 | XI-pellets1 | 1-4 | 24 | Liver | Azan & |
| | XI-pellets P2 | 17-20 | | | Haematoxylin/ |
| | Placebo 1 | 13-16 | | | Eosin Stains |
| | Placebo XI2 | 5-8 | | | |
| | XI-pellets 4 | 25-26 | | | |
| | XI-pellets P5 | 33-34 | | | |

Statistical Analyses: Data were subjected to statistical analysis using non-parametric test Kruskal-Wallis and Mann-Whitney (at 5% significance level).

Clinical Observations: Cage side observations during the 10 week treatment period were performed on all animals daily. Detailed clinical observations were performed weekly.

Body Weight: Body weights of all animals within the high fructose concentration during first 5 weeks regimens were significant higher (2.2-2.5 times) than body weights of all animals from groups fed by standard diet. Significant differences between groups XI-pellets 1-3 and Placebo 1-3 (HFRDXI-pellets/HFRD), XI-pellets 4-6 and Placebo 4-6 (SRDXI-pellets/SRD) were not observed.

Body weights of animals from groups XI-pellets 1/Placebo 1 fed HFRDXI-pellets/HFRD over the whole 10 week period continuously increased. During the first 5 week period, rat body weight gains were greater than that of the following period (week 6-10). The differences between final and initial body weight of these groups were 243.8 g and 206.3 g, respectively.

Body weights of animals from groups XI-pellets 4/Placebo 4 fed by SRDXI-pellets/SRD raised normally; with the exception of week 8 and week 9 in group Placebo 4 fed SRD and week 9 in group XI-pellets 4 fed SRDXI-pellets. During these weeks, the weights of the animals decreased slightly. Similarly, in these groups the body weight gains during the first 5 weeks were greater than in the following period. The differences between final and initial weights were 85 g and 92.5 g, respectively.

The differences between final and initial body weights in groups Placebo XI2/XI-pellets P2 treated 5 weeks with HFRDXI-pellets/HFRD and 5 weeks with HFRD/HFRDXI-pellets were 217.5 g and 193.8 g, respectively. The differences between final and initial body weights in groups Placebo XI5/XI-pellets P5 treated 5 weeks with SRDXI-pellets/SRD and 5 weeks with SRD/SRDXI-pellets were 90 g and 157.5 g, respectively. In animals Placebo XI5 group (SRD) during the period from week 8 to 9, and in animals XI-pellets P5 group (SRDXI-pellets) during week 9, the negative body gains were estimated.

Gradual weight elevation was observed throughout the study period with a higher rate in HFRDXI-pellets/HFRD rats. These animals showed an increased body weight compared to SRDXI-pellets/SRD, which was a consequence of high daily fructose consumption. Excess fructose consumption is a well-known cause of weight gain.

Food Consumption: Food consumption was recorded for all animals daily. Animals received diet ad libitum. During acclimatization, certified laboratory food was offered in recommended doses. Due to adaptation to new diet, the diet was served in a dose of 20 g/animal/day during the first week. The diet dose was increased to 25 g/animal/day during the second week, the daily dose was gradually increased (5 g step/day) until ad libitum doses were achieved. Average daily consumption was 20-32 g/rat/day in animals receiving HFRDXI-pellets/HFRD and 17-35 g/rat/day in animals receiving SRDXI-pellets/SRD.

Clinincal Chemistry Serum Parameters: Serum parameters (AST, ALT, GMT, glucose, cholesterol, TAG, albumin, and insulin) were measured 3 times during the study; before treatment, after week 5, and at the end of week 10. The levels of TAG, ALB and insulin were significantly changed in the animals from groups treated with HFRDXI-pellets/HFRD after week 5. Increases in ALB levels were statistically significant, but values were within control data for SD rats. Other parameters were not been significantly changed. Body weights of all high fructose regimen animals during the first 5 weeks were significantly higher than body weights of all animals from groups fed a standard diet.

Results of serum parameters after week 5 showed significantly increased levels of TAG (2.98±1.99/2.80±1.79) in all of XI-pellets 1-3/Placebo 1-3 animal groups receiving HFRDXI-pellets/HFRD versus animals from groups XI-pellets 4-6/Placebo 4-6 treated with SRDXI-pellets/SRD (0.68±0.21/0.48±0.12). Levels of TAG were increased 4- to 6-fold.

Animals from groups XI-pellets 1 and Placebo 1 were fed HFRDXI-pellets/HFRD for 10 weeks. These animals displayed increased TAG levels 3.5/4 times after week 5, and 2/1 times after week 10. TAG levels of group XI-pellets 1 compared with group Placebo 1 was higher 1.5 times after week 5, and very similar after week 10.

Animals from groups XI-pellets 2/Placebo XI2 and Placebo 2/XI-pellets P2 fed HFRDXI-pellets/HFRD for 5 weeks and HFRD/HFRDXI-pellets for following 5 weeks. These animals displayed increased TAG levels of 3/1.5 times after 5 weeks and 3/0 times after 10 weeks. At the end of the study, animals from group Placebo 2/XI-pellets P2, after a temporary increase during week 5, exhibited TAG values at the initial level.

Insulin values were significantly increased after week 5 (2.67±1.55/1.74±0.70) in all animals of XI-pellets 1-3/Placebo 1-3 groups treated with HFRDXI-pellets/HFRD against animals from groups XI-pellets 4-6/Placebo 4-6 treated with SRDXI-pellets/SRD (0.69±0.45/0.39±0.16). Insulin levels increased approximately 4/4.5 times.

Animals from groups XI-pellets 1 and Placebo 1 were fed HFRDXI-pellets/HFRD for 10 weeks. Insulin levels increased 3.2/1.8 times after week 5, and 2.4/2.8 times after week 10. The mean insulin level of group XI-pellets 1 compared with group Placebo 1 was 1.3 times higher after week 5, and 1.6 times lower after week 10.

Animals from group XI-pellets 2 fed HFRDXI-pellets for 5 weeks were fed with HFRD for following 5 weeks. The mean insulin level was increased over starting level after week 5, and significantly increased after week 10. Group Placebo 2 was fed HFRD for the initial 5 weeks, and HFRDXI-pellets for the following 5 weeks. Insulin values were significantly increased after weeks 5 and 10. The mean insulin value after week 10 was higher than after week 5.

TABLE 6
Clinical Chemistry - Serum
Clinical Chemistry - Serum

| | | | |
|---|---|---|---|
| | | Abbre- | Standard Operation Procedures& |
| Parameter | Unit | viation | Methods |

Enzymes

| | | | |
|---|---|---|---|
| Gama- | μkat/L | GGT | SPPB-00249-TOX, Enzymes |
| Glutamytransferase | | | |
| Aspartate | μkat/L | AST | |
| Aminotransferase | | | |
| Alanine | μkat/L | ALT | |
| Aminotransferase | | | |

Lipids

| | | | |
|---|---|---|---|
| Total Cholesterol | mmol/L | CHOL | SPPB-00244-TOX, Lipids |
| Triacylglycerols | mmol/L | TAG | |

Proteins

| | | | |
|---|---|---|---|
| Albumin | g/L | ALB | SPPB-00243-TOX, Proteins |

Saccharides

| | | | |
|---|---|---|---|
| Glucose | mmol/L | GLU | SPPB-00246-TOX, Saccharides |

Hormone

| | | | |
|---|---|---|---|
| Insulin | (ng/mL) | Insulin | Method 6240039: ELISA Assay |
| | | | for Determination of Rat |
| | | | Insulin VALR-00521-PH, |
| | | | Rat Insulin ELISA |

Clinincal Chemistry Liver Parameters: Liver parameters (IL-6, IL-10, Leptin, MCP-1, TNF-α, UAC and Fe) were measured after week 5 and after week 10 (end of study). Significant differences between levels of iron (Fe) and uric acid (UAC) in animals received HFRDXI-PELLET/HFRD as well as between groups with HFRDXI-PELLET/HFRD and SRDXI-PELLET/SRD after week 5 and week 10 were not observed. High variation in individual cytokine values was observed. Due to the low number of animals per group, a statistical evaluation was not performed.

The ratio of liver IL-10 and IL-6 values in rats fed HFRDXI-pellets/HFRD and rats fed SRDXI-pellets/SRD was 1.5 and 1.3 The ratio of leptin levels in rats, received HFRDXI-pellets/HFRD against rats received SRDXI-Pellets/SRD was greater than 17. Liver MCP-1 values were 1.3 times higher in rats fed HFRDXI-pellets/HFRD as compared to rats fed SRDXI-pellets/SRD. The mean TNF-α values were 1.5 times higher in rats fed HFRDXI-pellets/HFRD than in rats fed SRDXI-pellets/SRD. The significance of this comparison is reduced by the fact that TNF-α values in rat fed SRDXI-pellets/SRD were below or close to the limit of quantification.

TABLE 7
Clinical Chemistry - Liver
Clinical Chemistry - Liver

| | | Abbre- | Standard Operation Procedures& |
|---|---|---|---|
| Parameters | Unit | viation | Methods |
| Uric Acid | μmol/L | UAC | SPPB-00247-TOX, |
| | | | Nonprotein Nitrogenous |
| | | | [symbol] |
| Iron | μmol/L | Fe | SPPB-00245-TOX, Electrolyte |

Cytokines & Hormone

| | | | |
|---|---|---|---|
| Interleukin-6 | pg/mL | IL-6 | SPPA-00314-PH, |
| | | | Determination of IL- |
| | | | 6 in Rat Liver |
| Interleukin-10 | pg/mL | IL-10 | SPPB-000320-TOX, |
| [symbol] | [symbol] | [symbol] | Preparation of Samples- |
| Tumor Necrosis | pg/mL | TNF-alpha | [symbol] |
| Factor - alpha | | | |
| Leptin | pg/mL | LEP | |

[symbol]

indicates data missing or illegible when filed

Summary of Results

During the first 5 week period, rat body weight gains were greater than that of the week 6-10 period. No significant increases in the serum liver enzymes (aspartate aminotransferase, alanine aminotransferase, gamma glutamyltransferase) and cholesterol in the high fructose diet rats compared to the standard diet rats were observed. There were no differences among serum glycaemia within groups. Other markers evaluated, such as liver tissue content of uric acid (UAC) and Fe were similar in all groups. The levels of triacylglycerol (TAG) and albumin (ALB) were significantly changed in fructose fed rats. Possible effects of on TAG levels was observed only in rats fed with xylose-isomerase pellets during weeks 6-10. The levels of insulin were significantly changed in fructose-fed rats. A possible effect on insulin levels was registered in rats treated with high fructose and xylose-isomerase pellets during 10 weeks.

Rats fed diets containing a higher percentage of fructose displayed changes in tissue cytokine levels. MCP-1, IL-10, IL-6 and TNF-α levels were elevated in rats receiving high fructose diet versus rats receiving a standard diet. The highest increase was observed in leptin. Results of pathological evaluations demonstrated a positive effect of xylose-isomerase pellets during hepatic steatosis development of high fructose diet fed animals. This effect was reflected by a reduction in liver cell damage in rats from placebo group XI2 compared to xylose-isomerase pellet-fed group P2. This indicates that xylose-isomerase pellets had a preventive effect on development of hepatic steatosis for first five weeks. The therapeutic effect of xylose-isomerase pellets was manifested in a significant decrease of relative liver weight in animals of xylose-isomerase pellet-fed group 1 versus placebo group 1. Additionally, the absence of severe inflammation and fibrosis was detected.

### Summary of Clinical Chemistry Results

Non-alcoholic fatty liver disease (NAFLD) pathogenesis involves two stages. The first stage involved excessive accumulation of lipids in the cytoplasma of liver. The second stage includes injury due to oxidative stress and subsequent inflammatory reactions.

NAFLD is characterized by intracellular accumulation of lipids in the liver, hepatomegaly, increases in serum aminotransferases, and low-grade systemic inflammation. It has been suggested that NAFLD is a hepatic manifestation of insulin resistance syndrome (metabolic syndrome). Insulin resistance can be induced in animals by feeding them a high-fructose diet. Exposure of the liver to high quantities of fructose stimulates lipogenesis and TAG accumulation. A number of animal studies have demonstrated a correlation between high fructose diet and elevated serum glucose, insulin, and TAG levels.

No significant increases in serum glucose and liver enzymes (AST, ALT, and GMT) were observed in high fructose diet rats compared to standard diet rats. Excess fructose consumption is a well-established cause of weight gain. It is the results of higher serum TAG which is made from excess fructose in the liver (Zarfeshani A. et al). A possible effect on levels of TAG was observed only in animals from group XI-pellets P2. No changes in TAG were registered in animals group Placebo XI2. The trend of TAG results in animals from groups XI-pellets 1 and Placebo 1 was similar. After week 5, levels of TAG were increased, which decreased after week 10, although increase of TAG levels in group Placebo 1 was higher.

Insulin in animals fed high fructose diet (Placebo 1) was higher at 10 weeks than insulin in animals fed HF with XI-pellets (XI-pellets 1), which is a demonstrative effect of xylose-isomerase. No difference between XI-pellets P2 and Placebo XI2 groups was registered. No differences in hepatic UAC and iron concentrations were observed in rats treated with HFRDXI-pellets/HFRD as compared to rats fed a standard diet.

Cytokines and adipocytokines play a major role in inflammatory process throughout the body. The balance between pro- and anti-inflamatory acting cytokines/adipocytokines appears to play a key role in hepatic and systemic insulin action, as well as a role in the development of NAFLD.

The changes in IL-10 (anti-inflammatory cytokine) and IL-6 (multi-functional cytokine with a major role in mediating inflammation, insulin resistance and liver regeneration) between rats with HFRDXI-pellets/HFRD and rats fed SRDXI-pellets/SRD were registered. Leptin, a satiety hormone which regulates appetite, energy homeostasis, and glucose/lipid metabolisms, was increased in animals from groups with HFRDXI-pellets/HFRD against animals from groups of standard diet. TNF-α is recognized as the first cytokine that could induce insulin resistance (IR). An increased level of this cytokine was observed in animals of groups with HFRDXI-pellets/HFRD against animals with standard diet. Levels of MCP-1, mediator of inflammation, in rats with HFRDXI-pellets/HFRD were slightly elevated against rats with standard diet. Differences in levels of monitored cytokines between group with and without XI-pellets in HFRD were not observed.

### Pathology Results

Gross Pathology: Renal tubular mineralization (nephrocalcinosis) is a frequent finding in the SD rats. No macroscopical lesions were observed during necropsy of rats after 5 weeks of treatment. Expressive drawing of liver parenchyma was found in 1/4 rat (Placebo 1), in 2/4 rats (XI-pellets P2), in 1/4 rat (Placebo XI2).

Liver Weight: A statistically significant decrease in relative liver weight was noted in rats of XI-pellets 1 group as compared to Placebo 1 (Mann-Whitney test).

Histopathology

HF-Rat Diet after 5 Weeks of Treatment: The primary observance was the focal occurrence of microvesicles in liver cells cytoplasm in all 4 rats in XI-pellets 3 group and in Placebo 3 group. The occurrence of small inflammatory lesions was the same in 2/4 rats in both groups (XI-pellets 3 and Placebo 3). Sporadic monocellular necrosis were found in 2/4 rats in XI-pellets 3 group. Mostly small hyperaemia was present in all 4 rats from group XI-pellets 3 and in 2/4 rats from group Placebo 3.

Standard Rat Diet after 5 Weeks of Treatment: In the liver tissue taken from rats fed a standard diet, small or medium hyperaemia of sinusoid were observed in all animals from groups XI-pellets 6 and Placebo 6. Small numbers of microvesicles were found in 1/2 rat from Placebo 6 group and small inflammatory lesions were observed in 1/2 rat from XI-pellets 6 group.

HF-Rat Diet after 10 Weeks of Treatment: The most expressive micro- and macrovesicular steatosis were observed in 3/4 rats in group XI-pellets P2. Histopathological findings in two animals of this group were consistent with the results of macroscopic examination. The occurrence of vesicles in liver cells cytoplasm was observed in all rats fed HF diet (4/4 males), except group Placebo XI2 (3/4 males). Both micro- and macrovesicles were found in 2/4 rats in group XI-pellets 1; in 1/4 rat in Placebo 1, in 3/4 rats in XI-pellets P2 and in 2/4 rats in Placebo XI2. The expressive occurrence of steatosis in group XI-pellets P2 significantly differed from histopathological finding of steatosis in group Placebo XI2, where only sporadic fatty degeneration of liver cells was found. Fatty degenerative changes in liver cells were located usually in periportal areas. Sporadic or small inflammatory lesions were found in 1/4 rat (XI-pellets1), in 3/4 rats (Placebo 1, XI-pellets P2) and in all rats in group Placebo XI2. Sporadic monocellular necrosis was observed in 1/4 rat in group Placebo XI2 and in 1/4 rat in group XI-pellets P2.

Standard Rat Diet after 10 Weeks of Treatment: Small inflammatory lesions were found in 2/2 rats (Placebo 4, Placebo XI5).

### Discussion of Pathological Results

Steatosis in animals with HF-diet after 10 weeks was slightly larger and extensive than in animals after 5 weeks, except group Placebo XI2. Individual variance of histopathological lesions were noted among rats in each group. Differences in the development of steatosis were not observed between HF-diet and Placebo and XI-pellets treated groups at 5 weeks. However, after 10 weeks there were difference in the development of steatosis between groups XI-pellets P2 and Placebo XI2. The most expressive micro- and macrovesicular steatosis was observed in rats of group XI-pellets P2 as compared to group Placebo XI2, where no significant fatty degeneration of liver cells was found. Damage of liver cells did not cause reconstruction of the basic structure of the hepatic parenchyma as extension connective tissue components in any rat, which was confirmed by Azan staining method. In all animals fed a standard diet, the morphological structure of liver parenchyma was close to the normal liver for both time periods.

The development of the liver steatosis is influenced by many factors, including increased synthesis of fat in liver cells, decreased fat degradation, increased amount of fat transported to the liver, and decreased amount of fat transported from the liver. In the study with XI-pellets, the occurrence of steatosis in the liver was caused by combination of several factors in groups with HF-diet. Non-alcoholic fatty disease (NAFLD) is the most common cause of chronic liver disease and encompasses a number of diseases, from steatosis (lipid deposition) and non-alcoholic steatohepatitis (NAFLD inflammation) to cirrhosis (fibrosis) and liver failure. In the experiments and resulting analyses presented herein, a veritable method for the prevention and treatment of liver steatosis was demonstrated.

Anatomically, steatosis can take one of two forms depending on the size of the lipid vesicles: micro-vesicular steatosis is the condition in which fat is stored in multiple small vesicles within the hepatocyte cytoplasm, whereas macro-vesicular steatosis refers to the condition in which fat is stored in a single large vesicle. In the experiments described herein, both forms of steatosis were found and usually were observed in the same rat. The occurrences of inflammatory lesions in liver were slight and were found in standard rat diet as well as HF diet fed rats. Small inflammatory changes were observed in groups XI-pellets treated and in Placebo groups too.

### CONCLUSIONS

Sprague-Dawley male rats were fed ad libitum a standard or high fructose diet for 5 and 10 weeks. The effect of test item (XI-pellets) administered in diet on selected parameters characterising the development of non-alcoholic fatty liver disease like triglyceride accumulation, cholesterol content elevation, steatosis of the liver, induction of inflammatory cytokines, hepatic iron, and insulin resistance was evaluated. Body weights of all animals fed a high fructose concentration diet during the first 5 weeks were significant higher than body weights of all animals from groups fed by standard diet. During the first 5 week period, rat body weight gains were greater than that of the week 6-10 period. The levels of TAG and ALB were significantly changed in fructose-fed rats. Possible effect of test item (XI-pellets) on TAG levels was registered only in rats treated with XI-pellets during week 6-10. No significant increases in the serum liver enzymes (AST, ALT, GMT) and cholesterol in the high fructose diet compared to the standard diet were observed. Other markers evaluated, such as liver tissue content of UAC and Fe were similar in all groups. The levels of insulin were significantly changed in fructose-fed rats. Possible effect of test item on insulin levels was registered in rats treated with HF with XI-pellets during 10 weeks. Feeding with diet containing a higher percentage of fructose caused small changes in tissue cytokine levels: IL-10, IL-6 and TNF-α. Leptine levels were elevated in rats receiving a high fructose diet versus rats receiving a standard diet. Pathological evaluation results demonstrate a positive effect of XI-pellets during the hepatic steatosis development in high fructose diet reflected by a reduction of liver cells damage in rats from group Placebo XI2 in compare comparison with XI-pellets P2. This reduction in liver cell damage demonstrates that XI-pellets had a preventive effect during the development of the hepatic steatosis for first five weeks. The therapeutic effect of XI-pellets was manifested as both a statistically significant decrease of relative liver weight in animals of group XI-pellets 1 versus Placebo 1, and an absence of severe inflammation and fibrosis in the former group.

The claims are not to be interpreted as including means-plus- or step-plus-function limitations, unless such a limitation is explicitly recited in a given claim using the phrase(s) “means for” or “step for,” respectively.

### REFERENCES

1. Ackerman, Z. et al. Fructose-Induced Fatty Liver Disease: Hepatic Effects of Blood Pressure and Plasma Triglyceride Reduction, Hypertension, 2005, 45, 1012-1018.
2. Galipeau, D. et al. Female Rats are Protected Against Fructose-induced Changes in Metabolism and Blood Pressure, Am J Physiol Hearth Circ Physiol, 200, 283, H2478-H2482.
3. Abel, T. et al. Safety and Efficacy of Combined Ezetimibe/Simvastatin Treatment and Simvastatin Monotherapy in Patients with Non-Alcoholic Fatty Liver Disease, Med Sci Monit 2009, 15, MS6-MS11.
4. Catta-Preta, M. et al. A Critical Analysis of Three Quantitative Methods of Assessment of Hepatic Steatosis in Liver Biopsies, Virchows Arch 2011, 459, 477-485.
5. Zarfeshani A. et al. Evaluating Fructose Diet to Induce Hyperglycemia and its Inflamatory Complications in Rats, Pakistan Journal of Nutrition 2012, 11, 21-26.
6. Ku\check{c}era et al. The Effect of Rat Strain, Diet composition and Feeding Period on the Development of a Nutritional Model of Non-Alcoholic Fatty Liver Disease in Rats, Physiological Research/Academia Scientiarum Bohemoslovaca 2010, 60, 317-328.
7. Gikins M. L. A. et al. Clinical Laboratory Parameters for Crl: CD (SD) Rats, Charles River Laboratoties, March 2006.
8. Dische, Z. and Borenfreund, E. A New Spectrohpotometric Method for the Detection and Determination of Keto Sugars and Trioses, J. Biol. Chem. 1951, 192, 583-587.
9. Mathiowitz, E. et al. Biologically erodable microspheres as potential oral drug delivery systems, Nature, 1997, 386, 410-414.
10. Hwang, S. J. et al. Gastric Retentive Drug-Delivery Systems, Critical Reviews in Therapeutic Drug Carrier Systems, 1998, 15, 243-284.
11. Stricker, H. Arzneiformen-Entwicklung: Feste Zubereitungen, Springer-Verlag Berlin Heidelberg 2003.
12. Lustig, R. H. Fructose: It's “Alcohol Without the Buzz”, American Society for Nutrition: Advances in Nutrition 2013, 4, 226-235.
13. Komericki, P. et al. Oral xylose isomerase decreases breath hydrogen excretion and improves gastrointestinal symptoms in fructose malabsorption—a double-blind, placebo-controlled study. Alimentary Pharmacology and Therapeutics 2012, 36, 980-987.
14. Lustig, R. H et al. The toxic truth about sugar. Nature 2012, 482, 27-29.
15. Takahashi, Y. et al. Animal models of nonalcoholic fatty liver disease/nonalcoholic steatohepatitis. World Journal of Gastroenterology 2012, 18, 2300-2308.
16. Carrell, H. L. et al. X-ray analysis of D-xylose isomerase at 1.9 A: native enzyme in complex with substrate and with a mechanism-designed inactivator. Proceedings of the National Academy of Sciences 1989, 86, 4440-4444.
17. Suzuki, Y. et al. High-Pressure Acceleration of the Growth Kinetics of Glucose Isomerase Crystals. The Journal of Physical Chemistry B 2005, 109, 3222-3226.
18. Dunlop, K. V. and Hazes, B. A modified vapor-diffusion crystallization protocol that uses a common dehydrating agent, Acta Crystallographica Section D 2005, 61, 1041-1048.
19. Vilonen, K. M. et al. Enhanced Glucose to Fructose Conversion in Acetone with Xylose Isomerase Stabilized by Crystallization and Cross-Linking. Biotechnology Progress 2004, 20, 1555-1560.
20. Ramagopal, U. A. et al. SAD manganese in two crystal forms of glucose isomerase. Acta Crystallographica Section D 2003, 59, 868-875.
21. Vallejo-Becerra, et al. Immobilization of Recombinant Invertase (re-INVB) from Zymomonas mobilis on d-Sorbitol Cinnamic Ester for Production of Invert Sugar. Journal of Agricultural Food Chemistry 2008, 56, 1392-1397.
22. Wenzel, et al. Crosslinking of Dam methyltransferase with S-adenosyl-methionine FEBS Letters 1991, 280, 147-151.
23. Bhosale, S. G. et al. Molecular and industrial aspects of glucose isomerase, Microbial Reviews 1996, 60, 280-300.
24. Pillinger, M. H. and Abeles, A. M. Such Sweet Sorrow: Fructose and the Incidence of Gout, Current Rheumatology Reports 2010, 12, 77-79.

1. A method of treating or preventing non-alcoholic fatty liver disease in a subject comprising:
administering to the subject a composition comprising crystalline xylose-isomerase (EC 5.3.1.5) which originates from a microorganism of the family of Streptomycetaceae co-crystallized with a magnesium salt and at least one bivalent metal salt selected from the group consisting of cobalt salt, zinc salt, iron salt, and copper salt,
wherein the composition comprises a molar ratio of the bivalent metal salt selected from the group consisting of cobalt salt, zinc salt, iron salt, and copper salt to xylose-isomerase ranging from 3:1 to 7:1; and
wherein the composition further comprises a molar ratio of the magnesium salt to xylose-isomerase ranging from 5:1 to 200:1.

2. The method of claim 1, wherein the composition is administered in an enteric form.

3. The method of claim 2, wherein the enteric form is further defined as an enteric coated pellet, enteric coated tablet, enteric coated capsule, enteric coated granule, or enteric coated powder.

4. The method of claim 1, wherein the magnesium salt in the composition has a molar ratio to xylose-isomerase ranging from 5:1 to 25:1.

5. The method of claim 1, wherein the magnesium salt is MgCl₂, MgSO₄, MgCO₃, Mg(HCO3)2, or Mg(C₄H₂O₄).

6. The method of claim 1, wherein the xylose-isomerase is present in microcapsules, nanoparticles, or liposomes.

7. The method of claim 1, wherein the xylose-isomerase of microbial origin originates from Streptomyces rubiginosus.

8. The method of claim 1, wherein the composition is a pharmaceutical composition, a food supplement, a dietetic food, a medicinal product, a feeding stuff, a supplementary feeding stuff or a dietetic feeding stuff.

9. A method of treating hepatic steatosis in a subject comprising:
administering to the subject a composition comprising crystalline xylose-isomerase (EC 5.3.1.5) which originates from a microorganism of the family of Streptomycetaceae co-crystallized with a magnesium salt and at least one bivalent metal salt selected from the group consisting of cobalt salt, zinc salt, iron salt, and copper salt,
wherein the composition comprises a molar ratio of the bivalent metal salt selected from the group consisting of cobalt salt, zinc salt, iron salt, and copper salt to xylose-isomerase ranging from 3:1 to 7:1; and
wherein the composition further comprises a molar ratio of the magnesium salt to xylose-isomerase ranging from 5:1 to 200:1.

10. A method of reducing liver weight in a subject suffering from hepatic lipid accumulation comprising:
administering to the subject a composition comprising crystalline xylose-isomerase (EC 5.3.1.5) which originates from a microorganism of the family of Streptomycetaceae co-crystallized with a magnesium salt and at least one bivalent metal salt selected from the group consisting of cobalt salt, zinc salt, iron salt, and copper salt,
wherein the composition comprises a molar ratio of the bivalent metal salt selected from the group consisting of cobalt salt, zinc salt, iron salt, and copper salt to xylose-isomerase ranging from 3:1 to 7:1; and
wherein the composition further comprises a molar ratio of the magnesium salt to xylose-isomerase ranging from 5:1 to 200:1.

11. A method of preventing or reducing obesity in a subject comprising:
administering to the subject a composition comprising crystalline xylose-isomerase (EC 5.3.1.5) which originates from a microorganism of the family of Streptomycetaceae co-crystallized with a magnesium salt and at least one bivalent metal salt selected from the group consisting of cobalt salt, zinc salt, iron salt, and copper salt,
wherein the composition comprises a molar ratio of the bivalent metal salt selected from the group consisting of cobalt salt, zinc salt, iron salt, and copper salt to xylose-isomerase ranging from 3:1 to 7:1; and
wherein the composition further comprises a molar ratio of the magnesium salt to xylose-isomerase ranging from 5:1 to 200:1.
